(19) United States
(12) Patent Application Publication
(10) Pub. No.: US 2023/0210743 A1
(43) Pub. Date: Jul. 6, 2023

(54) DITHIONITE SHELF-STABLE SWEETENERS

(71) Applicant: CAO Group, Inc., West Jordan, UT (US)

(72) Inventor: Steven D. Jensen, South Jordan, UT (US)

(73) Assignee: CAO Group, Inc., West Jordan, UT (US)

(21) Appl. No.: 17/568,709

(22) Filed: Jan. 4, 2022

(51) Int. Cl. A61K 8/49; A61K 8/23; A61Q 11/00

(52) U.S. Cl. CPC A61K 8/49; A61K 8/23; A61Q 11/00

(57) ABSTRACT

A reductive composition, suitable for the dental industry, which utilizes sodium dithionite may be provided when it is combined with a sweetener selected from the salts of acesulfame. Sodium and potassium acesulfame show effective resistance to the reductive capacity of sodium dithionite. Other dithionite molecules, such as potassium dithionite, may be used.

### FIELD OF THE INVENTION

The present invention relates to the field of dentistry and more particularly relates to the use of reduction resistant sweeteners in tooth whitening compounds to create novel products.

### BACKGROUND OF THE INVENTION

Traditional tooth whitening compounds utilize oxidation reactions to whiten teeth, usually in the form of introducing peroxides to the enamel surface. These peroxide-based teeth whitening systems whiten by oxidizing the extrinsic and intrinsic stains found in teeth by removing electrons from the chromophores of stains, rendering them white. It should be noted that oxygen is not necessary for this process, however oxygen is notorious for initiating it, thus the element provides the name for the process. Hydrogen peroxide and its derivatives are stable in acidic pH environments and become increasingly unstable as the pH nears neutrality (pH 7). Bulk hydrogen peroxide is usually stored at a pH below 2. Acidic teeth whitening systems are detrimental to tooth enamel as they etch and dissolve tooth structure. Therefore, it is imperative that peroxide based whitening systems have at least a neutral pH. This is one of the main shelf-life instability factors with respect to peroxide-based tooth whiteners and the reason these compositions degrade over a short period of time. However, these whitening compounds also oxidize tooth enamel, resulting in tooth etching. As such, oxidative whitening of teeth is significantly regulated outside the United States and Canada.

Reduction reactions could also provide whitening effects. Reduction is the opposite reaction of oxidation in that electrons are added to a molecule and is often seen in conjunction with oxidation reactions (together being called redox reactions). Reduction of the chromophores of intrinsic and extrinsic enamel stains also renders them white. However, powerful reducing agents present an interesting challenge with respect to reactivity and compatibility when formulating various compositions. What usually occurs is an immediate reaction that results in a permanently reduced ingredient. For example, sodium dithionite (also known as sodium hydrosulfite, shown in the FIG. 1) is a powerful reducing agent commonly used in dying processes, water treatment, gas purification, photography, and other industries. It has not been previously used in dental whitening products. Sodium dithionite is highly reactive in the presence of water, quickly decomposing to thiosulfates and bisulfates when so exposed.

Like any oxidizing whitening composition, a reductive composition will have a noted unpleasant taste when used as a dental whitening agent. Therefore, a sweetener should be added to the composition to make the composition more palatable. When dithionite solutions, including those of either sodium or potassium dithionite, interact with sweeteners, such as saccharine or sugars, they will react until the sweetener is exhausted into insignificance, all the while exhausting a portion of the dithionite and reducing the composition's efficacy. This presents a problem for the preservation and delivery of an active ingredient such as sodium dithionite in a consumer product. Therefore, there is a need to find effective sweeteners that resist chemical reduction so that when in the presence of a reducing agent, like sodium dithionite, the reducing agent is preserved and measurable within the composition when the composition is to be used and the composition remains palatable for human use.

The present invention is a stable composition formulated from a combination of a reduction agent and sweeteners to provide a consumer dental whitening product. The present invention represents a departure from the prior art in that the compositions formulated using the teachings of the present invention allow for the use of highly reductive agents in wider industries, such as dentistry.

### SUMMARY OF THE INVENTION

The salts of acesulfame are surprisingly resistant to the reduction potential of dithionite. Aqueous compositions made with these sweeteners resist dithionite decomposition even in formulas that contain substantial water, which increases the reactivity and decomposition of dithionite. The sweetening capacity of the salts of acesulfame remains intact even in water and produce a shelf-stable sweetened product that is deliverable to a customer with sufficient active ingredient to be effective. As such, the salts of acesulfame may be used as a sweetening agent to create a palatable composition of sodium dithionite in the presence of water while producing a consumer whitening product.

In view of the foregoing disadvantages inherent in the known types of dental whitening products, an improved dental whitening composition may provide a chemically reductive dental composition utilizing a salt of dithionite that meets the following objectives: that the composition not contain peroxide, is substantially stable in alkaline pH And provides a palatable taste to encourage compliance by the end user. As such, a new and improved reductive composition may comprise a blend of salts of dithionite with salts of acesulfame to accomplish these objectives.

The more notable features of the invention have thus been outlined in order that the more detailed description that follows may be better understood and in order that the present contribution to the art may better be appreciated. Additional features of the invention will be described hereinafter and will form the subject matter of the claims that follow.

Many objects of this invention will appear from the following description and appended claims, reference being made to the accompanying drawings forming a part of this specification wherein like reference characters designate corresponding parts in the several views.

Before explaining at least one embodiment of the invention in detail, it is to be understood that the invention is not limited in its application to the details of construction and the arrangements of the components set forth in the following description or illustrated in the drawings. The invention is capable of other embodiments and of being practiced and carried out in various ways. Also, it is to be understood that the phraseology and terminology employed herein are for description and should not be regarded as limiting.

As such, those skilled in the art will appreciate that the conception, upon which this disclosure is based, may readily be utilized as a basis for the designing of other structures, methods, and systems for carrying out the several purposes of the present invention. It is important, therefore, that the claims be regarded as including such equivalent constructions as far as they do not depart from the spirit and scope of the present invention.

### BRIEF DESCRIPTION OF THE DRAWINGS

To describe the way the above-recited and other advantages and features of the invention can be obtained, a more particular description of the invention briefly described above will be rendered by reference to specific example embodiments thereof which are illustrated in the appended drawings. Understanding that these drawings depict only typical embodiments of the invention and are therefore not to be considered as limiting of its scope, the invention will be described and explained with additional specificity and detail using the accompanying drawings.

FIG. 1 is a drawing of a sodium dithionite molecule (prior art).

FIG. 2 is a drawing of a potassium acesulfame molecule (prior art).

FIG. 3 drawing of a sodium saccharin molecule (prior art).

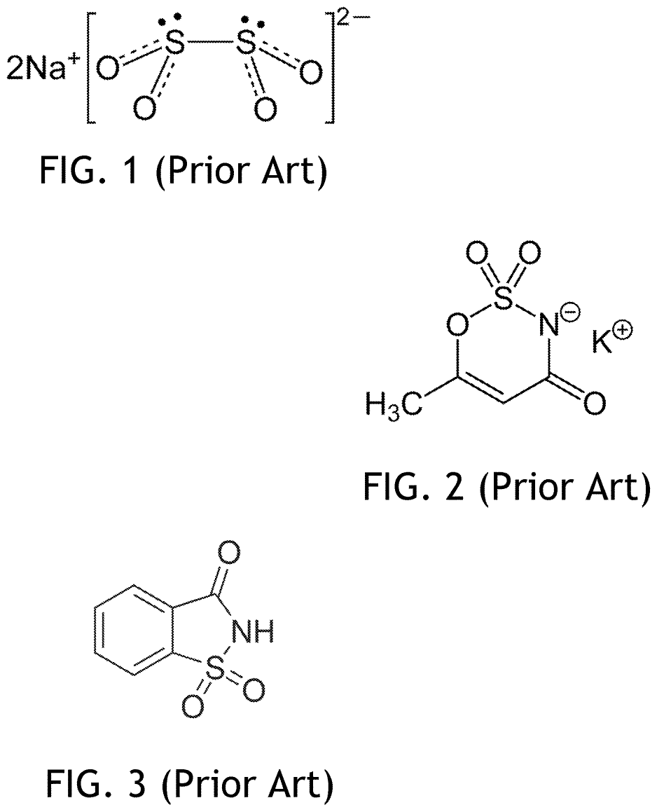

### DESCRIPTION OF THE PREFERRED EMBODIMENT

Preferred embodiments of the chemically reductive compositions are herein described. It should be noted that the articles “a,” “an,” and “the,” as used in this specification, include plural referents unless the content clearly dictates otherwise.

When dithionite solutions interact with sugars and/or artificial sweeteners such as sucralose or aspartame, they are quickly reduced and are no longer sweet. However, the salts of acesulfame (FIG. 2), particularly acesulfame potassium, which are commercially available, are compatible with dithionite; especially in compositions with a water content of less than 20%. Acesulfame is surprisingly resistant to the reduction potential of dithionite, even in the presence of water. This is a surprise since acesulfame is structurally like sodium saccharin, which is unstable with respect to dithionite and is no longer sweet within a few days (FIG. 3). Though functionally similar, and with similar moieties, it would be believed that acesulfame and saccharin would behave in an analogous manner chemically; yet this is not the case. Acesulfame retains its sweetness when combined with dithionite, even in the presence of water and at alkaline pH (above 7). Therefore, creating dental whitening compositions with salts of dithionite, such as sodium or potassium dithionite, with salts of acesulfame, such as acesulfame potassium or sodium acesulfame, create a palatable whitening composition that is remarkably shelf stable. While the named salts are preferred, other salts of dithionite or acesulfame may be utilized for similar, though possibly lesser, effect.

Although the present invention has been described with reference to preferred embodiments, numerous modifications and variations can be made and still the result will come within the scope of the invention. The described embodiments are to be considered in all respects only as illustrative and not restrictive. No limitation with respect to the specific embodiments disclosed herein is intended or should be inferred. Therefore, the scope of the invention is indicated by the appended claims rather than by the foregoing description. All changes which come within the meaning and range of equivalency of the claims are to be embraced within their scope.

What is claimed is:

1. A chemically reductive composition comprising a dithionite molecule interspersed in a medium with a salt of acesulfame.

2. The chemically reductive composition of claim 1, the dithionite molecule being chosen from the set of dithionite molecules consisting of sodium dithionite and potassium dithionite.

3. The chemically reductive composition of claim 1, the salt of acesulfame being selected from the set of the salts of acesulfame consisting of sodium acesulfame and potassium acesulfame.

4. A chemically reductive composition comprising dithionite and a salt of acesulfame.
